(19) United States
(12) Patent Application Publication
(10) Pub. No.: US 2017/0086687 A1
(43) Pub. Date: Mar. 30, 2017

(54) BIOSIGNAL MEASURING DEVICE AND BIOSIGNAL MEASURING SYSTEM

(71) Applicant: Kabushiki Kaisha Toshiba, Tokyo (JP)

(72) Inventor: Shigeo Imai, Chiba Chiba (JP)

(21) Appl. No.: 15/066,440

(22) Filed: Mar. 10, 2016

(30) Foreign Application Priority Data
Sep. 30, 2015 (JP) 2015-194441

(51) Int. Cl. A61B 5/024

(52) U.S. Cl. CPC A61B 5/02416

(57) ABSTRACT

According to embodiments, a biosignal measuring device includes a driver, an electric signal acquirer, a voltage signal generator, and an amplifier. The driver drives one or more light emitting elements to emit an optical signal having a light intensity that varies periodically. The electric signal acquirer acquires an electric signal corresponding to the amount of light of the optical signal reflected off the inside of a biological body, or corresponding to the amount of light of the optical signal transmitted through the biological body. The voltage signal generator outputs a voltage signal from which a frequency component which is equal to or lower than a predetermined frequency contained in the acquired electric signal has been removed. The amplifier amplifies the voltage signal and outputs an amplified signal.

### CROSS-REFERENCE TO RELATED APPLICATIONS

This application is based upon and claims the benefit of priority from Japanese Patent Application No. 2015-194441, filed on Sep. 30, 2015; the entire contents of which are incorporated herein by reference.

### FIELD

Embodiments of the present invention relate to a biosignal measuring device and a biosignal measuring system.

### BACKGROUND

PPG (Photoplethysmogram) measurement devices are known as an example of heart-rate measuring devices. A PPG measurement device measures a pulse wave using an LED (Light Emitting Diode) that emits light toward, for example, a blood vessel, and a photodiode that receives light transmitted through or reflected off the blood vessel. The output signal of the photodiode contains the pulse wave in the form of biological information. The pulse wave is detected by, for example, amplifying the output signal because the pulse signal component is very small.

However, the above output signal contains not only a pulse wave component but also a low-frequency noise component such as an environmental light component. Moreover, the level of the pulse wave component contained in the output signal is very low, and thus when the output signal is amplified, the pulse wave component is buried in the low-frequency noise component and may not be measured.

An object to be achieved by the present embodiment is to provide a biosignal measuring device that can measure biological information with high accuracy, and a biosignal measuring system.

### BRIEF DESCRIPTION OF THE DRAWINGS

FIG. 1 is a block diagram illustrating a schematic configuration of a biosignal measuring system according to a first embodiment;

FIG. 2A is a diagram illustrating the level of a voltage signal and the level of an amplified signal, in the absence of capacitors;

FIG. 2B is a diagram illustrating the level of the voltage signal and the level of the amplified signal, in the presence of the capacitors;

FIG. 3 is a block diagram illustrating the configuration of a biosignal measuring device according to a modification of the first embodiment;

FIG. 4 is a block diagram illustrating a schematic configuration of a biosignal measuring system according to a second embodiment;

FIG. 5 is a signal waveform chart for illustrating a demodulating process performed by a demodulator illustrated in FIG. 4; and

FIG. 6 is a block diagram illustrating the configuration of a biosignal measuring device according to a modification of the second embodiment.

### DETAILED DESCRIPTION

Embodiments will now be explained with reference to the accompanying drawings. The present invention is not limited to the embodiments.

### First Embodiment

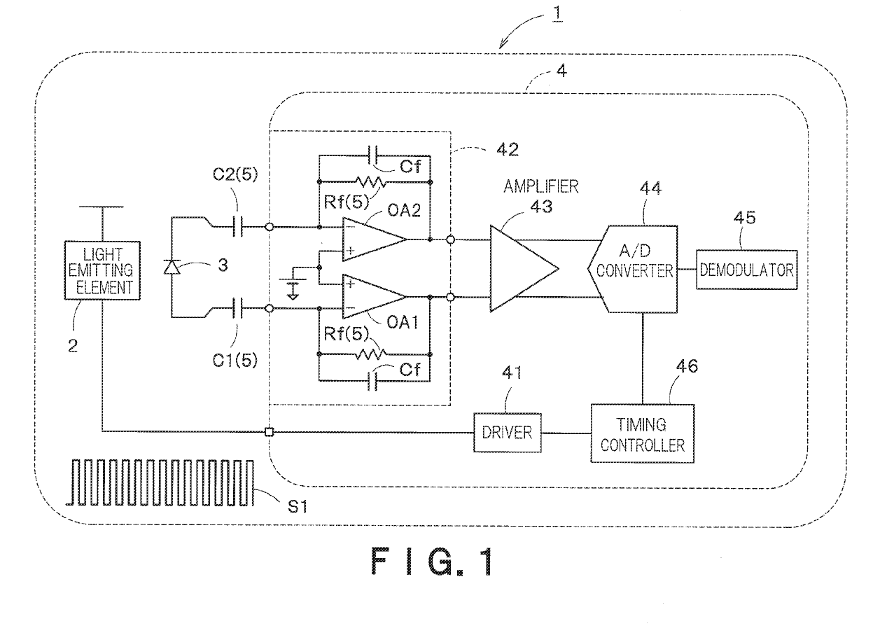

FIG. 1 is a block diagram illustrating a schematic configuration of a biosignal measuring system according to a first embodiment. As illustrated in FIG. 1, a biosignal measuring system 1 according to the present embodiment comprises a light emitting element 2, a light receiving element 3, and a biosignal measuring device 4.

The light emitting element 2 emits an optical signal S1 toward in a biological body (a blood vessel in the present embodiment). The light intensity of the optical signal S1 periodically varies, as illustrated in FIG. 1. The frequency of the optical signal S1 is, for example, at about a few kHz which is sufficiently higher than the frequency of a biological signal which is a few to a few tens of Hz.

In the present embodiment, the light emitting element 2 is composed of an LED that emits, for example, green light. However, the light emitting element 2 may be a light emitting element of a type other than that of LED. In addition, the luminescent color (the wavelength of light) of the light emitting element 2 may be the other colors (wavelengths) such as red and infrared.

In addition, in the present embodiment, the optical signal S1 is emitted from one light emitting element, but the optical signal S1 may be generated using a plurality of light emitting elements. More concretely, the optical signal S1 may be generated by causing a first light emitting element and a second light emitting element to alternately emit light, the first light emitting element having a high light intensity, the second light emitting element having a light intensity higher than that of the first light emitting element.

The light receiving element 3 receives a reflected optical signal that is the optical signal S1 emitted from light emitting element 2 and reflected off the inside of the biological body. Note that when the light of light emitting element 2 is red, the light receiving element 3 receives a transmitted optical signal that is transmitted through the biological body. In such a manner, the optical signal S1 emitted from the light emitting element 2 is amplitude-modulated by being reflected off the inside of the biological body or being transmitted through the biological body, and the amplitude-modulated optical signal is received by the light receiving element 3. In addition, the light receiving element 3 outputs a current signal that corresponds to the amount of light of the reflected optical signal or the transmitted optical signal that is received by the light receiving element 3. The current signal contains a pulse wave component, and a low-frequency noise component (a DC component) of environmental light and the like. In the present embodiment, the light receiving element 3 is composed of a photodiode. However, the light receiving element 3 may be a light receiving element of a type other than that of photodiode.

The biosignal measuring device 4 comprises a driver 41, a current-voltage converter 42, an amplifier 43, an A/D converter 44, a demodulator 45, and a timing controller 46. Each component will be described.

The driver 41 generates a drive signal for the light emitting element 2 to emit the optical signal S1 from the light emitting element 2.

The current-voltage converter 42 comprises operational amplifiers OA1 and OA2, resistance elements Rf, and capacitors Cf. The non-inverting input terminal of the operational amplifier OA1 and the non-inverting input terminal of the operational amplifier OA2 are connected to a constant voltage source. Note that, the current-voltage converter 42 comprises two single-ended operational amplifiers OA1 and OA2 in the present embodiment but may comprise a fully-differential operational amplifier, which is provided with a pair of output terminals.

The inverting input terminal of the operational amplifier OA1 is connected to the anode of the light receiving element 3 via a capacitor C1. Meanwhile, the inverting input terminal of the operational amplifier OA2 is connected to the cathode of the light receiving element 3 via a capacitor C2. The resistance elements Rf are provided between the inverting input terminals arid the output terminals of the operational amplifiers OA1 and OA2, respectively. The capacitors Cf are electronic components for smoothing a signal and for the stable operation of the operational amplifiers OA1 and OA2 and are connected to the resistance element Rf in parallel.

To the current-voltage converter 42 configured as described above, a current signal is input from the light receiving element 3 through the capacitors C1 and C2. The current signal is converted into a voltage signal and is output from the output terminals of OA1 and OA2. Then, a frequency component which is equal to or lower than a predetermined frequency contained in the current signal are removed by a first filter 5, which is composed of the capacitors C1 and C2 and the resistance elements Rf.

In the present embodiment, the first filter 5 functions as a high-pass filter that removes a frequency component which is lower than that of the optical signal S1 contained in the current signal, in other words, the first filter 5 functions as a high-pass filter that removes a low-frequency noise component of environmental light and the like. Note that the capacitors C1 and C2 constituting the first filter 5 is provided outside the biosignal measuring device 4 but may be provided inside the biosignal measuring device 4.

The amplifier 43 amplifies the voltage signal output from the current-voltage converter 42 and outputs the amplified signal. In the present embodiment, the amplifier 43 is composed of a PGA (Programmable Gain Amplifier) but may be composed of an amplifier of the other kind.

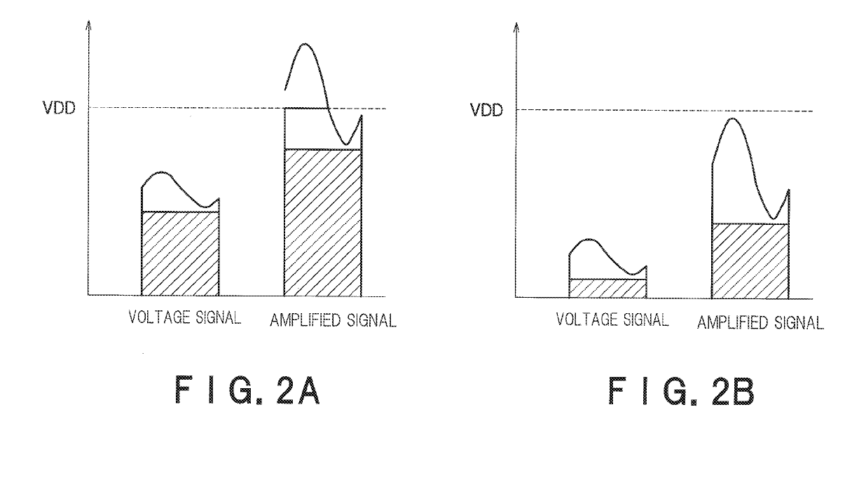

Now, referring to FIG. 2, there will be described a voltage signal input to the amplifier 43 and an amplified signal output from the amplifier 43. FIG. 2A is a diagram illustrating the level of the voltage signal and the level of the amplified signal, in the absence of the capacitors C1 and C2. FIG. 2B is a diagram illustrating the level of the voltage signal and the level of the amplified signal, in the presence of the capacitors C1 and C2. In FIG. 2A and FIG. 2B, hatched areas each represent the level of low-frequency noise component.

Without the capacitors C1 and C2, the current-voltage converter 42 converts a current signal output from the light receiving element 3 into a voltage signal as it is. Therefore, as illustrated in FIG. 2A, the level of the low-frequency noise component contained in the voltage signal is high. As a result, when the gain of the amplifier 43 is high, the level of the signal exceeds a power supply voltage VDD, causing signal clipping. Consequently, high-frequency pulse wave components contained in the voltage signal fail to be detected with fidelity.

On the other hand, with the capacitors C1 and C2 as in the present embodiment, when the current-voltage converter 42 converts a current signal into a voltage signal, the low-frequency noise component is substantially removed by the capacitors C1 and C2 and the resistance elements Rf. Therefore, as illustrated in FIG. 2B, the level of the low-frequency noise component contained in the voltage signal becomes very low. Therefore, the amplifier 43 can amplify a voltage signal within a range in which signal clipping does not occur and thus can amplify and detect a high-frequency pulse wave component contained in the voltage signal with a high gain.

Referring back to FIG. 1, the A/D converter 44 performs digital conversion on an amplified signal output from the amplifier 43 and Outputs the digital signal. In the present embodiment, the sampling frequency of the A/D converter 44 is twice or more the frequency of the optical signal S1.

The demodulator 45 performs digital processing on the digital signal output from the A/D converter 44 to detect a pulse wave component contained in the digital signal. As described above, an optical signal received by the light receiving element 3 is a signal which is generated by amplitude-modulating the optical signal S1 of the light emitting element 2. The current-voltage converter 42 converts an optical signal received by the light receiving element 3 into a voltage signal, and furthermore the A/D converter 44 performs digital conversion, and thereafter the demodulator 45 performs demodulation to detect a pulse wave component which is biological information. The timing controller 46 controls timing with which the A/D converter 44 operates, such that the A/D converter 44 performs digital conversion in synchronization with the optical signal S1.

There will be described below the operation performed by the biosignal measuring system 1 according to the above-described present embodiment.

First, the driver 41 drives the light emitting element 2. The light emitting element 2 thereby emits an optical signal S1 toward the biological body. The optical signal S1 is amplitude-modulated in the biological body and received by the light receiving element 3 as a reflected optical signal that is reflected off the inside of the biological body, or as a transmitted optical signal that is transmitted through the biological body. The light receiving element 3 outputs a current signal that corresponds to the amount of light of the reflected optical signal or the amount of light of the transmitted optical signal.

The above current signal is input to the current-voltage converter 42 through the capacitors C1 and C2, and the current-voltage converter 42 converts the current signal into a voltage signal. Then, as described above, a low-frequency noise component is removed by the first filter 5 which is composed of the capacitors C1 and C2 and the resistance elements Rf.

The voltage signal output from the current-voltage converter 42 is amplified by the amplifier 43 with a predetermined gain. The amplifier 43 outputs an amplified signal that is the amplified voltage signal to the A/D converter 44.

The A/D converter 44 outputs a digital signal into which the amplified signal is digitally converted, to the demodulator 45. Finally, the demodulator 45 extracts, from the digital signal, a digital value that corresponds to a pulse wave component, and a pulse wave is thereby detected.

According to the above-described biosignal measuring system 1 of the present embodiment, the amplifier 43 can amplify a voltage signal from which a low-frequency noise component has been removed by the capacitors C1 and C2 and the current-voltage converter 42. Therefore, it is possible to secure a high gain, allowing a pulse wave to be measured with high accuracy.

In addition, in the present embodiment, since the light emitting element 2 emits an optical signal S1 at a high frequency, it is possible to amplitude-modulate the optical signal S1 using a pulse wave. As a result, the frequency band of the optical signal S1 subjected to the amplitude modulation becomes a few kHz. In contrast, the frequency band of a low-frequency noise component is a few Hz. Therefore, the difference between the frequency band of a puke wave and the frequency band of a low-frequency noise component becomes very large. This enables a low-frequency noise component to be easily removed without using a high-precision filter. This means that it is possible to optically detect a pulse wave component without being influenced by low-frequency noise components of environmental light and the like.

Note that in the biosignal measuring device 4 according to the present embodiment, at least the driver 41, the current-voltage converter 42, and the amplifier 43 may be provided in a chip, as components of a semiconductor device.

In addition, in the present embodiment, the current-voltage converter 42 comprises an electric signal acquirer that acquires an electric signal corresponding to the amount of light of a signal received by the light receiving element 3 (a reflected optical signal, a transmitted optical signal), and a voltage signal generator that outputs a voltage signal from which components at a predetermined frequency or lower contained in the acquired electric signal are removed. Concretely, the inverting input terminals of the operational amplifiers OA1 and OA2 constitute the electric signal acquirer, and the output terminals of the operational amplifiers OA1 and OA2 and the resistance elements Rf constitute the voltage signal generator.

However, the electric signal acquirer and the voltage signal generator are not limited to the configuration in which they are integrated as the current-voltage converter 42, but may be separated. Furthermore, the electric signal acquired by the electric signal acquirer is not only a current signal output from the light receiving element 3, but also a voltage signal into which the current signal is subjected to voltage conversion. That is, a process of converting the optical signal received by the light receiving element 3 into a voltage may be performed outside the biosignal measuring device 4. In this case, the biosignal measuring device 4 will receive a voltage signal that corresponds to the optical signal received by the light receiving element 3. Since the biosignal measuring device 4 comprises the electric signal acquirer that acquires the received voltage signal as the above-described electric signal, and the voltage signal generator that outputs a voltage signal from which components at a predetermined frequency or lower contained in the acquired electric signal are removed, it is possible to detect a pulse wave component by the same operation of process as that in FIG. 1.

### (Modification 1)

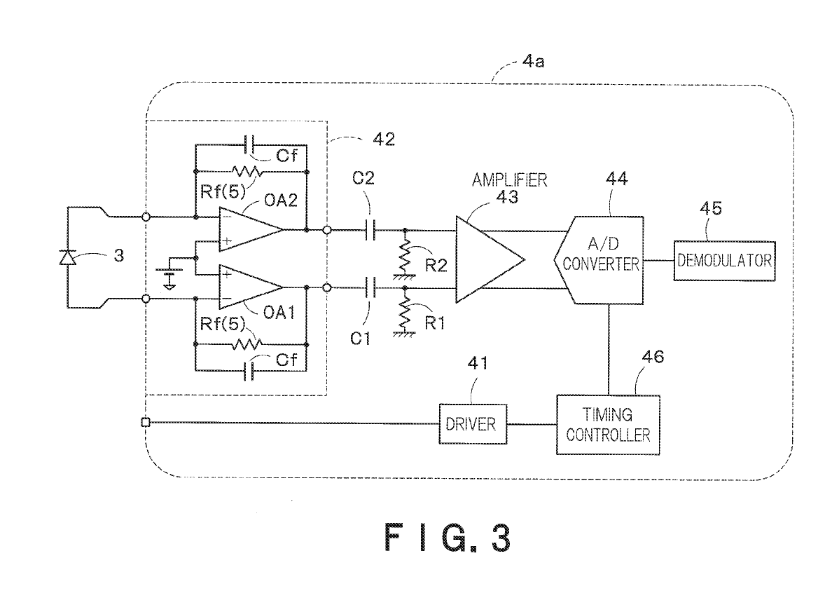

FIG. 3 is a block diagram illustrating the configuration of a biosignal measuring device according to a modification of the first embodiment.

As illustrated in FIG. 3, in a biosignal measuring device 4a according to the present modification, the capacitors C1 and C2 are connected in series to the output terminals of the operational amplifiers OA1 and OA2, respectively. In addition, one end of a resistance R1 is connected on the connection path between the capacitor C1 and the amplifier 43, and the other end thereof is grounded. Furthermore, one end of a resistance R2 is connected on the connection path between the capacitor C2 and the amplifier 43, and the other end thereof is grounded.

In the present modification, a current signal output from the light receiving element 3 is directly input to the current-voltage converter 42. The current-voltage converter 42 converts the current signal into a voltage signal and outputs the voltage signal. Then, the voltage signal contains a low-frequency noise component. However, the low-frequency noise component is removed by the capacitors C1 and C2 and the resistances R1 and R2 before input to the amplifier 43. That is, in the present modification, the current-voltage converter 42 functions as the above electric signal acquirer, and the capacitors C1 and C2 and the resistances R1 and R2 functions as the above-voltage signal generator.

Also in the above-described present modification, the amplifier 43 can amplify a voltage signal from which a low-frequency noise component has been removed by the capacitors C1 and C2 and the resistances R1 and R2. Therefore, it is possible to secure a high gain, allowing a pulse wave component to be measured with high accuracy.

### Second Embodiment

A biosignal measuring system according to a second embodiment will be described. The same components as those of the biosignal measuring system 1 according to the above-mentioned first embodiment are denoted by the same reference numerals, and will not be described in detail.

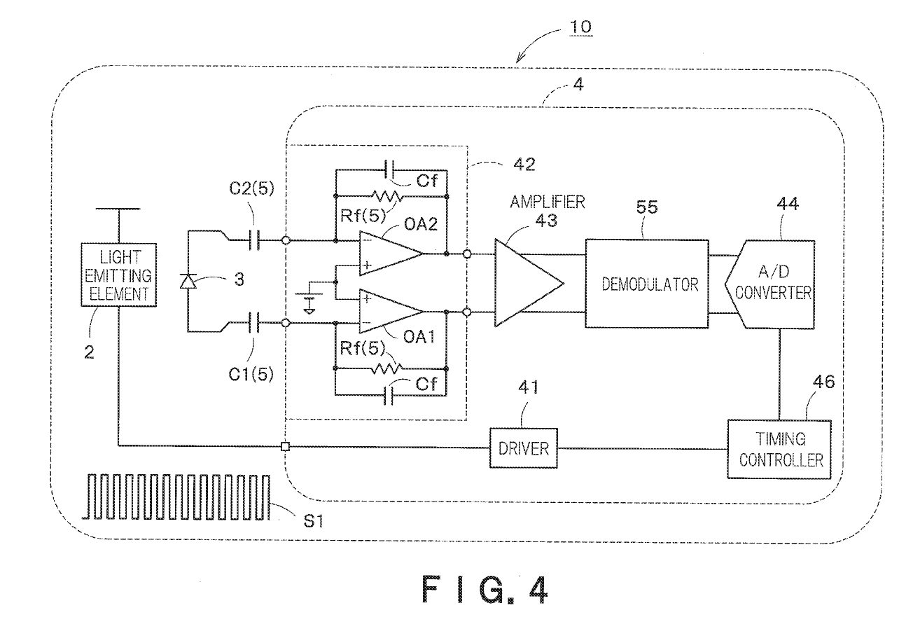

FIG. 4 is a block diagram illustrating a schematic configuration of the biosignal measuring system according to the second embodiment. As illustrated in FIG. 4, a biosignal measuring system 10 according to the present embodiment is different from the biosignal measuring system 1 according to the first embodiment in that the biosignal measuring system 10 comprises a demodulator 55 in place of the demodulator 45.

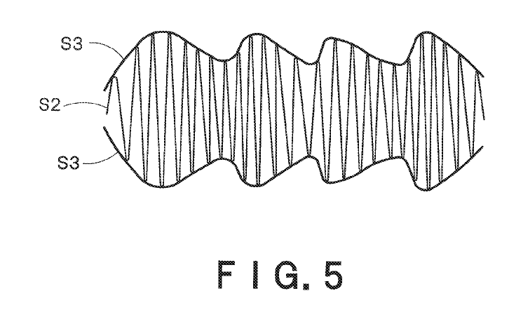

The demodulator 55 is provided between the amplifier 43 and the A/D converter 44. The demodulator 55 performs a demodulating process of detecting a pulse wave from an amplified signal amplified by the amplifier 43. The demodulating process performed by the demodulator 55 will be described below with reference to FIG. 5. FIG. 5 is a signal waveform chart for illustrating the demodulating process performed by the demodulator 55.

An amplified signal S2 illustrated in FIG. 5 is an analog signal obtained by subjecting a high-frequency optical signal S1 to amplitude modulation using a pulse wave. The demodulator 55 comprises, for example, a peak hold circuit. This peak hold circuit functions as a low-frequency converter that extracts a peak component from the amplified signal S2, namely, envelope information on the amplified signal S2, and outputs a low-frequency signal S3 at a frequency lower than that of the amplified signal S2.

The low-frequency signal S3 is a signal corresponding to the pulse wave signal. That is, the demodulator 55 performs operation of detecting the pulse wave signal from the amplified signal S2. Then, the pulse wave signal is converted into a digital signal by the A/D converter 44.

According to the biosignal measuring system 10 of the above-described present embodiment, as with the first embodiment, the amplifier 43 can amplify a voltage signal from which a low-frequency noise component has been removed. Therefore, it is possible to secure a high gain, allowing a pulse wave to be measured with high accuracy.

In particular, in the present embodiment, the demodulator 55 detects a pulse wave signal before the amplified signal S2 is subjected to digital conversion, lowering the frequency of the to be input to the A/D converter 44. Therefore, it is possible to lower the sampling frequency of the A/D converter 44. This enables the A/D converter 44 to perform digital processing on a pulse wave signal even if the sampling frequency of the A/D converter 44 is lower than twice the frequency of the optical signal S1. That is, it is possible to perform digital processing on a pulse wave signal even if the A/D converter 44 does not have a high functionality.

### (Modification 2)

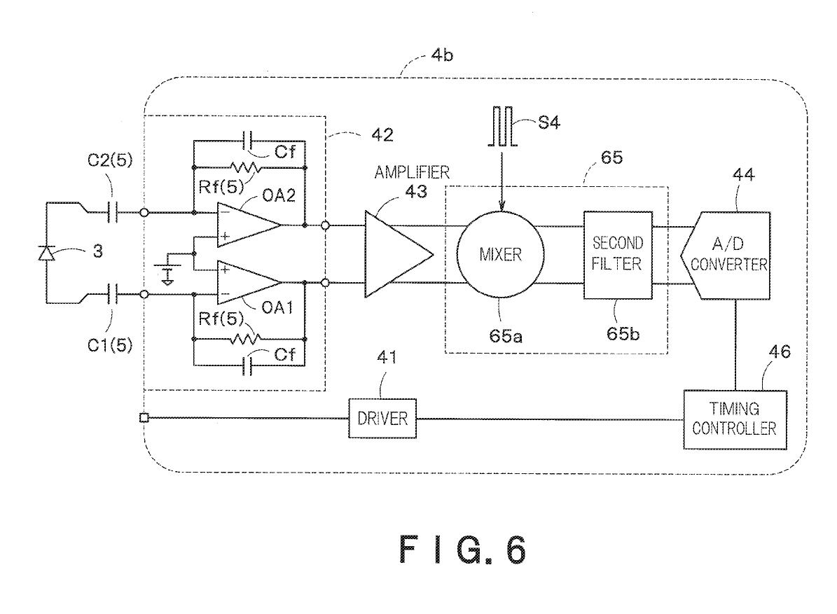

FIG. 6 is a block diagram illustrating the configuration of a biosignal measuring device according to a modification of the second embodiment. As illustrated in FIG. 6, a biosignal measuring device 4b according to the present modification comprises a demodulator 65. The demodulator 65 comprises a mixer 65a and a second filter 65b.

The mixer 65a combines the amplified signal S2 output from the amplifier 43 and a signal S4 having the same frequency as the frequency of the optical signal S1. The mixer 65a then outputs a first signal and a second signal. The first signal is a signal having a frequency that is a sum of the frequency of the amplified signal S2 and the frequency of the signal S4 (the frequency of the optical signal S1). In contrast, the second signal is a signal having a frequency that is the subtraction between the frequency of the amplified signal S2 and the frequency of the signal S4 (the frequency of the optical signal S1).

The second filter 65b removes the first signal to detect the second signal. The second signal is a signal generated by subtracting of a frequency the same as the frequency of the optical signal S1 from the frequency of the amplified signal S2. The amplified signal S2 contains a high-frequency component of the optical signal S1 and a low-frequency component of a pulse wave signal. Therefore, the subtraction the frequency of the optical signal S1 from the frequency of the amplified signal S2 makes the above low-frequency component left in the second signal. The second signal generated in such a manner, being at a low frequency, corresponds to the low-frequency signal S3 illustrated in FIG. 5. That is, the second signal is a signal corresponding to the pulse wave signal.

Also in the present modification, the demodulator 65 detects a pulse wave signal before the amplified signal S2 is subjected to digital conversion. Therefore, it is possible to perform digital processing on a pulse wave signal even if the sampling frequency of the A/D converter 44 is not high, in other words, the sampling frequency of the A/D converter 44 is lower than twice the frequency of the optical signal S1.

In the above-described embodiments and modifications, the descriptions are made about examples in which a signal output from the demodulator 45 or the A/D converter 44 is a measurement signal of a pulse rate. However, the above-described biosignal measuring devices 4, 4a, and 4b are applicable not only to the measurement of a pulse rate but also to, for example, the measurement of an oxygen saturation in blood. That is, a signal output from each biosignal measuring device can contain biological information on other than a pulse wave.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A biosignal measuring device comprising:
a driver that drives one or more light emitting elements to emit an optical signal having a light intensity that varies periodically;
an electric signal acquirer that acquires an electric signal corresponding to an amount of light of the optical signal reflected off the inside of a biological body, or corresponding to an amount of light of the optical signal transmitted through the biological body;
a voltage signal generator that outputs a voltage signal from which a frequency component which is equal to or lower than a predetermined frequency contained in the acquired electric signal has been removed; and
an amplifier that amplifies the voltage signal and outputs an amplified signal.

2. The biosignal measuring device according to claim 1, wherein
the electric signal acquired by the electric signal acquirer is a current signal,
the voltage signal generator comprises a resistance element, and
the voltage signal generator removes the frequency component that is equal to or lower than the predetermined frequency contained in the current signal by a first filter that is composed of the resistance element and a capacitor being connected to the electric signal acquirer, and outputs the voltage signal.

3. The biosignal measuring device according to claim 1, further comprising
a demodulator that detects biological information in the biological body based on the amplified signal.

4. The biosignal measuring device according to claim 3, further comprising
an A/D converter that performs digital conversion on the amplified signal and outputs a digital signal, wherein
the demodulator detects the biological information in the digital signal.

5. The biosignal measuring device according to claim 4, wherein a sampling frequency of the A/D converter is twice or more than a frequency of the optical signal.

6. The biosignal measuring device according to claim 3, wherein
the demodulator comprises a low-frequency converter that extracts a peak component of the amplified signal and that outputs a low-frequency signal having a frequency which is lower than the amplified signal, and
the biosignal measuring device further comprises an A/D converter that converts the low-frequency signal into the digital

7. The biosignal measuring device according to claim 6, wherein
the low-frequency converter comprises:
a mixer that combines the amplified signal and a signal having a frequency identical to a frequency of the optical signal emitted by the light emitting element, and that outputs a first signal and a second signal, wherein the first signal has a frequency that is a sum of the frequency of the amplified signal and of the frequency of the optical signal, and the second signal has a frequency that is a subtraction between the frequency of the amplified signal and the frequency of the optical signal; and
a second filter that removes the first signal and detects the second signal as the low-frequency signal.

8. The biosignal measuring device according to claim 1, wherein
the electric signal acquirer comprises a current-voltage converter that acquires a current signal as the electric signal and that converts the acquired current signal into a voltage signal having a frequency component which is equal to or lower than the predetermined frequency, and
the voltage signal generator comprises:
a capacitor connected to an output side of the current-voltage converter; and
a resistance element connected to the capacitor, wherein
the capacitor and the resistance element remove the frequency component which is equal to or lower than the predetermined frequency contained in the voltage signal converted by the current-voltage converter.

9. The biosignal measuring device according to claim 6, wherein
a sampling frequency of the A/D converter is lower than twice a frequency of the optical signal.

10. A biosignal measuring system comprising:
one or more light emitting elements that emit an optical signal to a biological body, the optical signal having a light intensity that varies periodically;
a light receiving element that receives the optical signal reflected off the inside of the biological body, or the optical signal transmitted through the biological body, arid that outputs a current signal;
a driver that drives the one or more light emitting elements;
a current-voltage converter that outputs a voltage signal from which a frequency component which is equal to or lower than a predetermined frequency contained in the current signal has been removed; and
an amplifier that amplifies the voltage signal and outputs an amplified signal.

11. The biosignal measuring system according to claim 10, further comprising:
a semiconductor device comprising the driver, the current-voltage converter, and the amplifier; and
a capacitor being connected to the light receiving element and the semiconductor device, wherein
the current-voltage converter comprises a resistance element being connected to the capacitor, and
the resistance element and the capacitor constitute a first filter that removes a frequency component which is equal to or lower than the predetermined frequency contained in the current signal.
